(19) United States
(12) Patent Application Publication
(10) Pub. No.: US 2026/0294246 A1
(43) Pub. Date: Oct. 1, 2026

(54) SAFETY CHECKING SYSTEM, CONTROL DEVICE, LIVING ROOM, AND SAFETY CHECKING METHOD

(71) Applicant: SEKISUI HOUSE, LTD., Osaka (JP)

(72) Inventors: Shouto IWATA, Osaka (JP); Buntoku MORI, Osaka (JP); Mina OKITSU, Osaka (JP); Hiroko OKEMOTO, Osaka (JP); Masae NAKAMOTO, Osaka (JP); Kazuhiko MATSUSHITA, Osaka (JP)

(21) Appl. No.: 18/993,411

(22) PCT Filed: Mar. 8, 2024
(86) PCT No.: PCT/JP2024/008949

§ 371 Date: Jan. 10, 2025

(30) Foreign Application Priority Data
Jul. 12, 2023 (JP) 2023-114719

(51) Int. Cl. A61B 5/00; A61B 5/024; A61B 5/08

(52) U.S. Cl. CPC A61B 5/002; A61B 5/024; A61B 5/0816; A61B 5/7282

(57) ABSTRACT

This invention prevents or suppresses erroneous sensing and an increase in a processing load that may occur in a system for checking whether a subject is safe. A safety checking system (1) according to an aspect of this invention includes: a first sensor (41) which senses, in a non-contact manner, biological information of a subject within a given sensing range; a second sensor (42) which detects, in a non-contact manner, the number of subjects in the sensing range of the first sensor; and a control section (30) which controls the first sensor (41) and the second sensor (42), the control section (30) causing, in a case where the second sensor (42) detects that only one subject is present within the sensing range, the first sensor (41) to measure biological information of the one subject.

### TECHNICAL FIELD

The present invention relates to a safety checking system, a control device, a living room, and a safety checking method.

### BACKGROUND ART

There are still many cases of acute diseases and accidents occurring at home. Further, in recent years, the aging population is growing and the number of people living alone is also increasing. Against this background, it is increasingly important to deal with accidents and acute diseases occurring at home.

Under the circumstances, Patent Literature 1, for example, discloses a building including a living space in which a resident lives and which is provided with a sensor, the building being configured to transmit a notification for prompting for checking of whether or not the resident is safe, if the sensor senses a preset abnormal state. Further, Patent Literature 2 discloses a body movement signal processing apparatus capable of detecting body movement of a subject in a bathroom.

### CITATION LIST

### Patent Literature

Patent Literature 1
Japanese Patent Application Publication, Tokukai, No. 2015-26146 (Publication date: Feb. 5, 2015)
Patent Literature 2
Japanese Patent, No. 6537701 (Publication date: Sep. 8, 2017)

### SUMMARY OF INVENTION

### Technical Problem

However, according to the above-described conventional techniques, there is a possibility that a sensor for measuring a respiratory rate and/or the like of a subject who is to be subjected to safety checking may erroneously sense a measurement item. Further, even when no subject exists in a sensing range, measurement of a respiratory rate and/or the like may be attempted with use of the above sensor, which increases a processing load of the system, disadvantageously.

An aspect of the present invention was made in consideration of the above problems, and has an object to prevent or suppress erroneous sensing and an increase in a processing load that may occur in a system for checking whether or not a subject is safe.

### Solution to Problem

In order to attain the above object, a safety checking system in accordance with an aspect of the present invention includes: a first sensor which senses, in a non-contact manner, biological information of a subject within a given sensing range; a second sensor which detects, in a non-contact manner, the number of subjects within the sensing range of the first sensor; and a control section which controls the first sensor and the second sensor, the control section being configured to cause, in a case where the second sensor detects that only one subject is present within the sensing range, the first sensor to measure biological information of the one subject.

In order to attain the above object, a control device in accordance with an aspect of the present invention is configured to control: a first sensor which senses, in a non-contact manner, biological information of a subject within a given sensing range; and a second sensor which detects, in a non-contact manner, the number of subjects within the sensing range of the first sensor, and in a case where the second sensor detects that only one subject is present within the sensing range, the control device causing the first sensor to measure biological information of the one subject.

In order to attain the above object, a living room in accordance with an aspect of the present invention includes: a first sensor which senses, in a non-contact manner, biological information of a subject within a given sensing range in the living room; a second sensor which detects, in a non-contact manner, the number of subjects within the sensing range of the first sensor; and a control device which controls the first sensor and the second sensor, the control device being configured to cause, in a case where the second sensor detects that only one subject is present within the sensing range, the first sensor to measure biological information of the one subject.

In order to attain the above object, a safety checking method in accordance with an aspect of the present invention includes: a sensing step of causing a first sensor provided in a living room to sense, in a non-contact manner, biological information of a subject within a given sensing range in the living room; a detection step of causing a second sensor provided in the living room to detect, in a non-contact manner, the number of subjects within the sensing range of the first sensor; and a measurement step of causing, in a case where the second sensor detects that only one subject is present within the sensing range, the first sensor to measure biological information of the one subject.

The safety checking systems in accordance with the aspects of the present invention may be realized by a computer. In such a case, the present invention encompasses: a control program for a safety checking system, the control program causing a computer to operate as the sections (software elements) of the safety checking system so that the safety checking system can be realized by the computer; and a computer-readable storage medium storing the control program therein.

### Advantageous Effects of Invention

In accordance with an aspect of the present invention, it is possible to prevent or suppress erroneous sensing and an increase in a processing load that may occur in a system for checking whether or not a subject is safe.

### BRIEF DESCRIPTION OF DRAWINGS

FIG. 1 is a view illustrating an overview of a safety checking system in accordance with an embodiment of the present invention.

FIG. 2 is a view illustrating an overview of the safety checking system.

FIG. 3 is a view illustrating an example of how a first sensor and a second sensor are provided.

FIG. 4 is a view illustrating an example of how first sensors and second sensors are provided.

FIG. 5 is a view illustrating an example of how first sensors and second sensors are provided.

FIG. 6 is a flowchart illustrating a flow of a process in the safety checking system.

FIG. 7 is a view illustrating an example of thresholds to be compared with a measurement result of a second sensor.

### DESCRIPTION OF EMBODIMENTS

### Embodiments

The following description will discuss, with reference to the drawings, details of an embodiment of the present invention. Although the embodiment discussed in the following description includes various limitations that are preferable from a technical perspective for putting the present invention into practice, the technical scope of the present invention is not limited to the embodiment below or the examples in the drawings.

### Overview

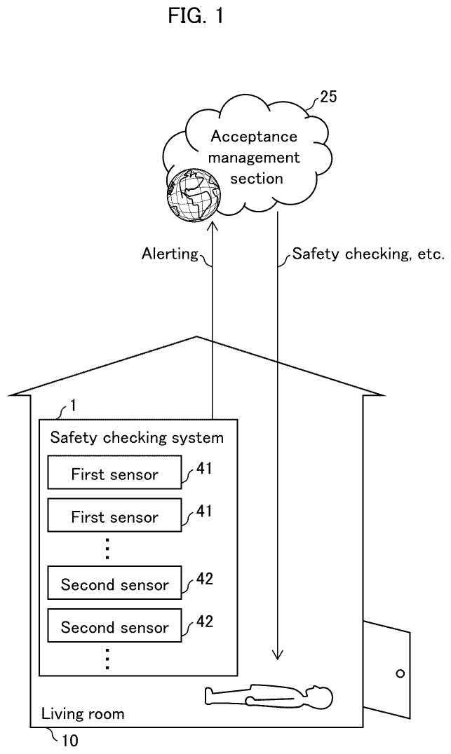

First, the following will describe, with reference to FIG. 1, an overview of a safety checking system 1 in accordance with the present embodiment. FIG. 1 is a view illustrating an overview of the safety checking system 1. As shown in FIG. 1, the safety checking system 1 is a system that (i) checks whether or not an abnormality occurs in a heart rate and/or the like of a resident and/or the like in a living room 10 in a residential building and (ii) issues, if an abnormality occurs, an alert indicative of occurrence of the abnormality to, e.g., an external device manipulated by an operator, for example.

In the description below, the safety checking system 1 is provided in the living room 10. However, this is not limitative. A part of or the whole of the system may be provided in, e.g., a facility such as a hospital or any space. In the description below, a target to be subjected to safety checking by the safety checking system 1 may sometimes be called a “subject”.

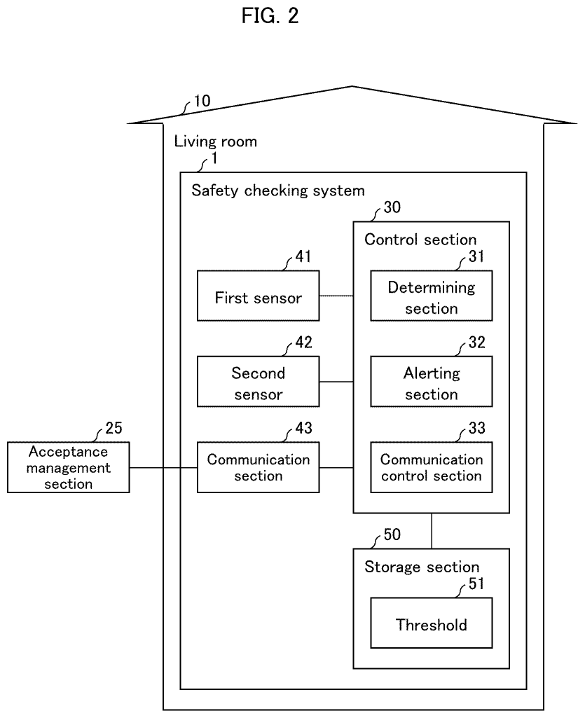

FIG. 2 is a view illustrating an overview of the safety checking system 1. As shown in FIG. 2, the safety checking system 1 includes a first sensor 41, a second sensor 42, a control section 30, a storage section 50, and a communication section 43.

The first sensor 41 obtains biological information by sensing, in a non-contact manner, the biological information of a subject within a sensing range. To be more specific, the first sensor 41 senses, in a non-contact manner, at least one of a heart rate, a respiratory rate, a body temperature, and a blood pressure of the subject. The present embodiment deals with an example in which the first sensor 41 senses and measures, in a non-contact manner, at least one of the heart rate and the respiratory rate of the subject within the sensing range, among the biological information. Note that the present disclosure also encompasses a configuration in which the first sensor 41 senses and measures other biological information in a non-contact manner. In the description below, the first sensor 41 is a Doppler sensor. However, the first sensor 41 is not limited to the Doppler sensor. For example, the first sensor 41 may be an infrared array sensor, a frequency modulated continuous wave (FMCW) radar, or the like.

The second sensor 42 detects, in a non-contact manner, the number of subjects within a detection range. In the following description, the second sensor 42 is a pyroelectric sensor. However, this is not limitative. The second sensor 42 may be any of other sensors capable of the above-described detection. In a case where the second sensor 42 is a pyroelectric sensor, the second sensor 42 carries out detection of a subject by sensing an infrared ray emitted from the subject.

A sensing range of the first sensor 41 and a detection range of the second sensor 42 may at least partially overlap each other, and one of the sensing range of the first sensor 41 and the detection range of the second sensor 42 may be included in the other. For example, the detection range of the second sensor 42 may be larger than the sensing range of the first sensor 41. The expression “one is included in the other” includes a case where the one and the other coincide with each other.

The sensing range of the first sensor 41 and the detection range of the second sensor 42 may be ranges extending in the same direction from their respective sensors. In other words, the first sensor 41 and the second sensor 42 may be provided to a same wall, a same ceiling, a same floor, a same built-in shelf, same furniture, or the like and may be configured to carry out emission in the same direction. Examples of a configuration which can be an alternative to the above configuration include a configuration in which one sensor is provided to a ceiling so as to face a floor and the other sensor is provided to the floor so as to face the ceiling.

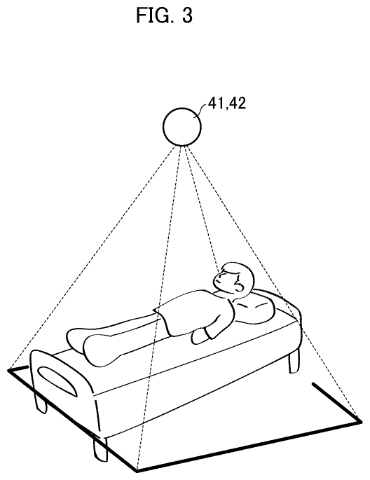

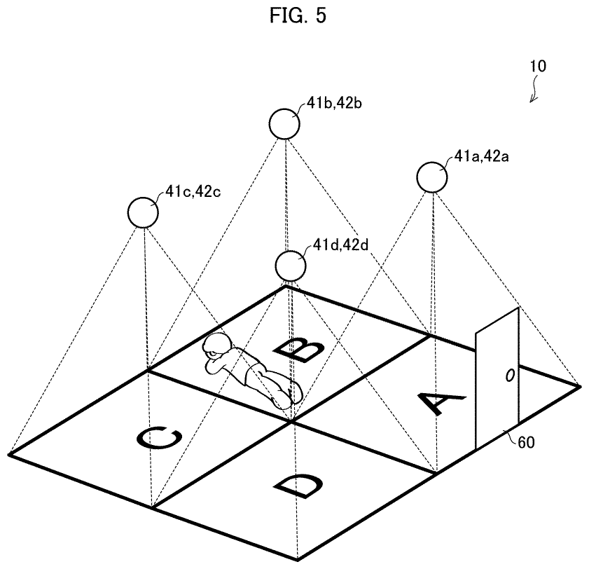

Each of FIGS. 3 to 5 is a view illustrating an example of how the first sensor 41 and the second sensor 42 are provided. In FIG. 5, the first sensor 41 is illustrated as first sensors 41a to 41d and the second sensor 42 is illustrated as second sensors 42a to 42d, for convenience of reference in the later-presented description.

In the example shown in each of FIGS. 3 to 5, the first sensor 41 and the second sensor 42 are provided to the ceiling so as to carry out radiation toward the floor. In each of FIGS. 3 to 5, the sensors are provided adjacent to each other so as to carry out radiation in the same direction, and the sensing range of the first sensor 41 and the sensing range of the second sensor 42 are identical to each other.

Further, at least either of the number of the first sensors 41 and the number of the second sensors 42 provided in the safety checking system 1 may be two or more, as shown in FIG. 1, etc. This is also true of other constituent elements in the safety checking system 1. Sensing ranges of a plurality of first sensors 41 or detection ranges of a plurality of second sensors 42 may partially overlap each other.

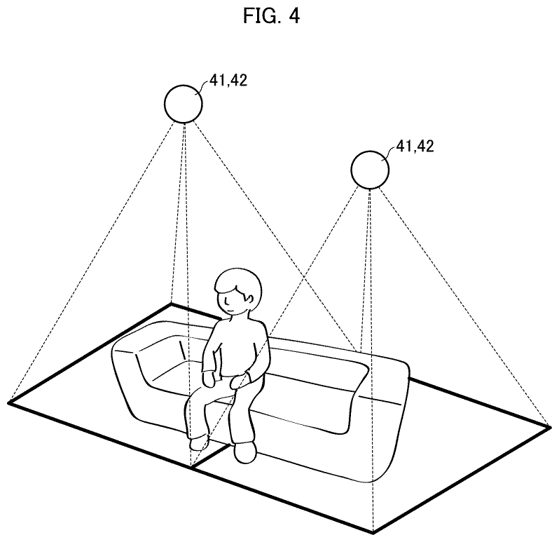

Each of FIGS. 4 and 5 shows, as an example, a configuration in which a plurality of first sensors 41 and a plurality of second sensors 42 are provided to a single room of the living room 10. In such a configuration, the plurality of sensors may be configured to cooperate with each other so as to make it possible to deal with even a case where the subject's body lies across two or more sensing ranges (detection ranges).

The control section 30 is a control device which controls the entire safety checking system 1 by controlling the first sensor 41, the second sensor 42, and the like, and also functions as a determining section 31, an alerting section 32, and a communication control section 33.

Further, in a case where the second sensor 42 detects that only one subject is present within the sensing range of the first sensor 41, the control section 30 carries out control for measuring at least one of a heart rate and a respiratory rate of the subject with use of the first sensor 41.

The determining section 31 carries out determination on sensing results obtained by the first sensor 41 and the second sensor 42. Details of the determination will be described later.

In a case where the determining section 31 determines occurrence of an abnormality in the subject, the alerting section 32 issues, as an alert, information indicative of it to an acceptance management section (external device) 25 via the communication section 43. Here, examples of the external device include a terminal device manipulated by an operator shown in FIG. 1 and a mobile terminal or the like possessed by a family member of the subject. The acceptance management section 25 may be operation carried out by an operator. Alternatively, the operation carried out by the operator may be partially or entirely replaced with machine learning by AI and/or the like. Data relating to acceptance dealt with by the operator has a large amount of information. Therefore, machine learning by AI and/or the like is useful to generate determination information. The communication control section 33 carries out control relating to communication processing by the communication section 43.

The storage section 50 is a storage device storing therein at least thresholds 51 which is to be compared with the measurement result of the first sensor 41. The storage section 50 may store therein information such as biological information (data relating to a physique such as an age, a gender, a height, and/or a weight as well as data indicative of a heart rate and/or the like) of the subject in a normal state and/or behavior information (information indicative of a behavior pattern according to a day of week, a time zone, and/or the like). The thresholds 51 may be calculated by the control section 30 by referring to at least either of the biological information and the behavior information of the subject. Further, the control section 30 stores, in the storage section 50, the information indicative of the heart rate or the respiratory rate of the subject measured by the first sensor 41. The communication section 43 is an interface for carrying out communication with an external device.

Another aspect different from that shown in FIG. 2 may be configured such that at least one of the control section 30, the storage section 50, and the communication section 43 may be included in at least either of the first sensor 41 and the second sensor 42. Further, the safety checking system 1 may additionally include, for example, an input section (not illustrated) which accepts a user's input of information stored in the storage section 50 and which is realized as, e.g., a button(s) of a keyboard or a touch panel. Alternatively, the information stored in the storage section 50 may be information which has been input via a mobile terminal such as a smartphone and which has been obtained by the control section 30 via the communication section 43.

### Flow of Process in Safety Checking System 1

Next, the following will describe, with reference to FIGS. 5 to 7, a flow of a process in the safety checking system 1.

First, the following will provide supplemental information of FIG. 5. In the living room 10 shown in FIG. 5, each of a sensing range of the first sensor 41a and a detection range of the second sensor 42a is an area A. Each of a sensing range of the first sensor 41b and a detection range of the second sensor 42b is an area B. Each of a sensing range of the first sensor 41c and a detection range of the second sensor 42c is an area C. Each of a sensing range of the first sensor 41d and a detection range of the second sensor 42d is an area D. In a case where the first sensors 41a to 41d are not particularly distinguished from each other, a part of or all of the first sensors 41a to 41d may sometimes be called a “first sensor 41”. In a case where the second sensors 42a to 42d are not particularly distinguished from each other, a part of or all of the second sensors 42a to 42d may sometimes be called a “second sensor 42”.

Further, an entrance 60 for entry into/exit from the living room 10 shown in FIG. 5 is provided only to an outer side of the area A. Thus, in order to get to one of the areas B to D from the outside of the living room 10, it is necessary to pass through the area A. Further, assume that a second sensor 42e (not illustrated) is additionally provided in the vicinity of the entrance 60, although provision of the second sensor 42e is not essential.

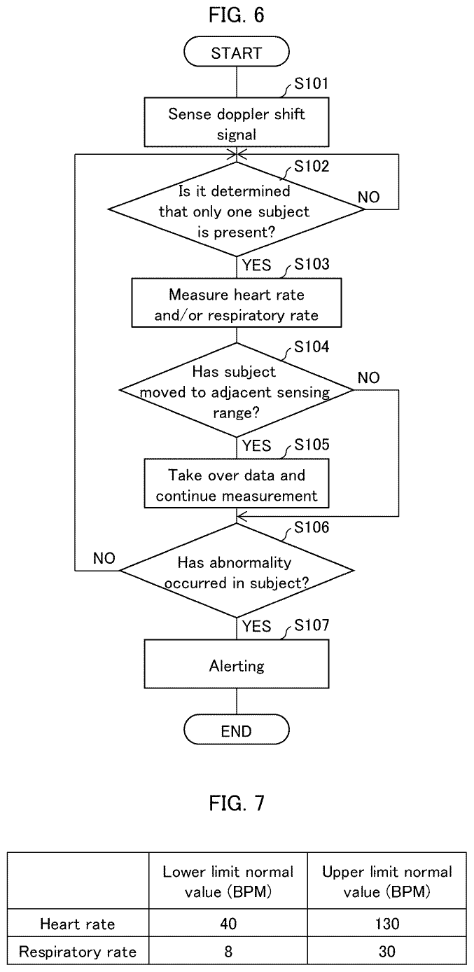

FIG. 6 is a flowchart illustrating a flow of a process in the safety checking system 1. The process illustrated in the flowchart in FIG. 6 starts when the second sensor 42e or another second sensor 42 detects a subject entering the living room 10.

The first sensors 41a to 41d start a process for emitting microwaves and sensing reflected microwaves (S101). Here, the reflected microwaves have a frequency with a Doppler shift occurring in accordance with movement of an object to which the microwaves are emitted. Note that, in this step, execution of the process of emitting microwaves is not essential. Alternatively, the process of emitting microwaves may be executed just before execution of a process in the later-described step S103.

Subsequently, the determining section 31 determines whether or not any of the second sensors 42 has detected that a subject is present within a sensing range of a corresponding one of the first sensors 41 (S102). If the determining section 31 determines that any of the second sensors 42 has carried out such detection (YES in S102, detection step), then the one of the first sensors 41 executes a process in step S103. If the determining section 31 determines that any of the second sensors 42 has not carried out such detection (NO in S102), the process in step S102 is continued until any of the second sensors 42 detects the subject.

If any of the second sensors 42 detects a plurality of subjects within the detection range, the process may not advance to step S103 and the process in step S102 may be continued. The process is carried out in this manner for the following reason. That is, in a case where a plurality of subjects stay close to each other, it is considered that the subjects can notice each other's abnormalities, if the abnormalities occur. Further, this can prevent or suppress erroneous measurement that may occur when the measurement is interfered with by a person coming close to a subject who is being measured. However, even in a case where a plurality of subjects are present in the living room 10, if the plurality of subjects are detected in respective different detection ranges of the second sensors 42, then the first sensors 41 may execute the process in step S103. Such a configuration is significant when the living room 10 is large enough and the second sensors 42 having detected the respective subjects are separated away from each other.

Subsequently, the first sensor 41 corresponding to the second sensor 42 having carried out the above detection refers to the above-described reflected microwaves and starts measuring a heart rate and a respiratory rate of the subject (S103, measurement step). Here, the first sensor 41 corresponding to the second sensor 42 means the first sensor 41 whose sensing range coincides with the detection range of the subject second sensor 42. For example, in a case where the second sensor 42a detects that only one subject is present within the sensing range of the first sensor 41a, the first sensor 41a starts measuring a heart rate and a respiratory rate of the subject. In this case, the first sensors 41b to 41d whose sensing ranges include no subject do not carry out measurement of a heart rate and a respiratory rate.

Subsequently, while the measurement of the heart rate and the respiratory rate is being carried out by the first sensor 41, the determining section 31 determines whether or not the second sensor 42 has detected movement of the subject to an adjacent sensing range (area) (S104). If the determining section 31 determines that the second sensor 42 has detected movement of the subject to the adjacent sensing range (YES in S104), then a process in step S105 will be executed. If the determining section 31 determines that the second sensor 42 has not carried out such detection (NO in S104), then a process in step S106 is executed.

Subsequently, the first sensor 41 corresponding to the sensing range from which the subject has moved ends the measurement of the heart rate and the respiratory rate, and a first sensor 41 corresponding to a sensing range to which the subject has moved starts measuring a heart rate and a respiratory rate of the subject (S105). For example, in a case where the subject has moved from the area A to the area B, the first sensor 41a ends measurement of a heart rate and a respiratory rate of the subject, and then the first sensor 41b takes over and executes the above measurement. The pieces of data measured by these first sensors 41 will be dealt with as a series of pieces of data.

In other words, in a case where a second sensor 42 corresponding to a first sensing range and a second sensor 42 corresponding to a second sensing range which is adjacent to the first sensing range detect that only one subject having been present in the first sensing range has moved to the second sensing range, the control section 30 stores, in the storage section 50, (i) data indicative of a heart rate and a respiratory rate of the subject measured by the first sensor 41 corresponding to the first sensing range and (ii) data indicative of a heart rate and a respiratory rate of the subject measured by the first sensor 41 corresponding to the second sensing range, as a series of pieces of data.

Further, the processes in steps S104 and S105 can be executed two or more times. That is, every time the subject moves to a detection range adjacent to the second sensor 42, the first sensor 41 which measures the heart rate and the respiratory rate is changed to a first sensor 41 corresponding to the second sensor 42 of question. The resulting pieces of measurement data are stored in the storage section 50 as a series of pieces of data.

Subsequently, the determining section 31 determines whether or not the measurement result of the first sensor 41 is within a value range defined by the thresholds 51 stored in the storage section 50 (hereinafter, this may sometimes simply be referred to as “within a value range”) (S106). FIG. 7 is a view illustrating an example of the thresholds 51. Here, “BPM (Beats Per Minute)” in FIG. 7 denotes the number of heart beats or the like per minute. For example, in the case of the heart rates illustrated in FIG. 7, if a heart rate per minute of the subject is not less than 40 and not more than 130, the determining section 31 determines that the measurement result of the first sensor 41 is within the value range; otherwise, the determining section 31 determines that the measurement result of the first sensor 41 is outside the value range defined by the thresholds 51 stored in the storage section 50 (hereinafter, this may sometimes simply be referred to as “outside a value range”). Further, in the case of the respiratory rates indicated as an example, if a respiratory rate per minute of the subject is not less than 8 and not more than 30, the determining section 31 determines that the measurement result of the first sensor 41 is within the value range; otherwise, the determining section 31 determines that the measurement result of the first sensor 41 is not within the value range but outside the value range.

As described above, the thresholds 51 which are to be compared with the measurement result of the second sensor 42 may be the one corresponding to at least one of normal biological information and normal behavior information of the subject. That is, FIG. 7 illustrates, as an example, a simplified configuration of the thresholds 51. This is not limitative. The determining section 31 may carry out determination by thresholds 51 varying depending on a characteristic of a subject, such as an age, a gender, or fitness habit.

If the determining section 31 determines that the measurement result of the first sensor 41 is outside the value range (YES in S106), then the alerting section 32 executes a process in step S107. If the determining section 31 determines that the measurement result of the first sensor 41 is within the value range (NO in S106), the process in step S102 is executed again.

As another aspect of step S106, the determining section 31 may be configured to (i) refer to a plurality of measurement results that the first sensor 41 has obtained for a latest given time period and (ii) determine, regarding at least either of the heart rate and the respiratory rate of the subject, whether or not a percentage of measurement results determined as not being within the value range is not less than a given percentage. According to this configuration, if the determining section 31 determines that the percentage of the measurement results determined as not being within the value range is not less than the given percentage (YES in S106), then the alerting section 32 executes the process in step S107. If the determining section 31 determines that this percentage is less than the given percentage (NO in S106), the process in step S102 is executed again.

Subsequently, the alerting section 32 issues, as an alert, information indicative of occurrence of an abnormality in the subject to the acceptance management section (external device) 25 via the communication section 43 (S107). Then, the acceptance management section 25 may automatically inquire of a residential building including the living room 10 provided with the safety checking system 1 as to whether or not the subject is safe, for example.

Further, the process illustrated in the flowchart shown in FIG. 6 may be executed until the second sensor 42e and the second sensors 42a to 42d detect that all subjects have exited from the living room 10. In a case where it is determined that all the subjects have exited from the living room 10, the first sensor 41 may end the process of emitting microwaves and sensing reflected microwave.

According to the method explained with reference to the flowchart shown in FIG. 6, it is possible to prevent or suppress erroneous sensing and an increase in a processing load that may occur in the method for checking whether or not the subject is safe. Further, in a case where no abnormality occurs in the subject, it is possible to acknowledge subject's life rhythm, e.g., information indicating which one of living rooms in the residential building the subject stays for a longest period of time.

### Additional Remarks

It is not necessarily required that the safety checking system 1 include the communication section 43 and the control section 30 function as the alerting section 32. For example, the safety checking system 1 may be configured such that, in a case where the control section 30 determines that an abnormality occurs in a subject, this is notified by, e.g., a buzzer.

Further, the safety checking system 1 may be configured to carry out measurement by the first sensor 41 and determination based on a resulting measurement result for only either of a heart rate and a respiratory rate.

The safety checking system 1 may be configured such that, in a case where the measurement result of the first sensor 41 and any of the above-discussed thresholds 51 are identical to each other, it is determined that the measurement result is within the value range or outside the range.

The living room 10 may be provided with a switch for enabling or disabling a function of at least either of the first sensor 41 and the second sensor 42 provided in the living room 10. For example, in a case where the subject does exercise in the living room 10, the subject can manipulate the switch to temporarily disable the functions of the sensors, thereby allowing the safety checking system 1 to prevent or suppress erroneous determination of occurrence of an abnormality in the heart rate or the respiratory rate of the subject.

Aspects of the present invention can also be expressed as follows:
A safety checking system in accordance with a first aspect of the present invention includes: a first sensor which senses, in a non-contact manner, biological information of a subject within a given sensing range; a second sensor which detects, in a non-contact manner, the number of subjects within the sensing range of the first sensor; and a control section which controls the first sensor and the second sensor, the control section being configured to cause, in a case where the second sensor detects that only one subject is present within the sensing range, the first sensor to measure biological information of the one subject.

According to the above configuration, only after the second sensor detects that only one subject is present within the sensing range of the first sensor, the first sensor carries out the measurement. This makes it possible to prevent or suppress erroneous sensing and an increase in a processing load that may occur in a system for checking whether or not a subject is safe.

A safety checking system according to a second aspect of the present invention may be configured such that, in the first aspect, in a case where the second sensor detects that only the one subject having been present within the sensing range has exited from the sensing range, the first sensor is caused to end the measurement of the biological information of the one subject.

According to the above configuration, when the subject is no longer present within the sensing range, the first sensor ends the measurement. This contributes to prevention or suppression of erroneous sensing and an increase in the processing load.

A safety checking system according to a third aspect of the present invention may be configured such that, in the first or second aspect, in a case where a second sensor corresponding to a first sensing range and a second sensor corresponding to a second sensing range which is adjacent to the first sensing range detect that only the one subject having been present in the first sensing range has moved from the first sensing range to the second sensing range, (i) data of biological information of the one subject measured by a first sensor corresponding to the first sensing range and (ii) data of biological information of the one subject measured by a first sensor corresponding to the second sensing range are dealt with as a series of continuous pieces of data.

According to the above configuration, even when a user moves between a plurality of sensing ranges, it is possible to continue a series of measurements by the first sensor.

A safety checking system according to a fourth aspect of the present invention may be configured such that, in any of the first to third aspects, the second sensor has a detection range larger than the sensing range of the first sensor.

According to the above configuration, the configuration in which the biological information of the subject is measured when the user is present within the sensing range of the first sensor can be realized by using a smaller number of second sensors than the number of first sensors.

A safety checking system according to a fifth aspect of the present invention may be configured such that, in any of the first to fourth aspects, the first sensor measures at least either of a heart rate and a respiratory rate of the one subject.

According to the above configuration, it is possible to check whether or not the subject is safe on the basis of the heart rate or the respiratory rate of the subject.

A control device according to a sixth aspect of the present invention is configured to control: a first sensor which senses, in a non-contact manner, biological information of a subject within a given sensing range; and a second sensor which detects, in a non-contact manner, the number of subjects within the sensing range of the first sensor, and, in a case where the second sensor detects that only one subject is present within the sensing range, the control device causing the first sensor to measure biological information of the one subject.

The above configuration contributes to realization of the above-described safety checking system.

A living room according to a seventh aspect of the present invention includes: a first sensor which senses, in a non-contact manner, biological information of a subject within a given sensing range in the living room; a second sensor which detects, in a non-contact manner, the number of subjects within the sensing range of the first sensor; and a control device which controls the first sensor and the second sensor, the control device being configured to cause, in a case where the second sensor detects that only one subject is present within the sensing range, the first sensor to measure biological information of the one subject.

According to the above configuration, it is possible to realize a living room that provides effects similar to those of the above-described safety checking system.

A safety checking method according to an eighth aspect of the present invention includes: a sensing step of causing a first sensor provided in a living room to sense, in a non-contact manner, biological information of a subject within a given sensing range in the living room; a detection step of causing a second sensor provided in the living room to detect, in a non-contact manner, the number of subjects within the sensing range of the first sensor; and a measurement step of causing, in a case where the second sensor detects that only one subject is present within the sensing range, the first sensor to measure biological information of the one subject.

The above configuration provides effects similar to those of the above-described safety checking system.

### Software Implementation Example

Control blocks (particularly, the determining section 31, the alerting section 32, and the communication control section 33 of the control section 30) included in the safety checking system 1 can be realized by a logic circuit (hardware) provided in an integrated circuit (IC chip) or the like or can be alternatively realized by software.

In the latter case, the safety checking system 1 includes a computer that executes instructions of a program that is software realizing the foregoing functions. The computer, for example, includes at least one processor and a computer-readable storage medium storing the program. An object of the present invention can be achieved by the processor of the computer reading and executing the program stored in the storage medium. Examples of the processor encompass a central processing unit (CPU). Examples of the storage medium encompass a “non-transitory tangible medium” such as a read only memory (ROM), a tape, a disk, a card, a semiconductor memory, and a programmable logic circuit. The computer may further include a random access memory (RAM) or the like in which the program is loaded. Further, the program may be made available to the computer via any transmission medium (such as a communication network and a broadcast wave) which allows the program to be transmitted. Note that an aspect of the present invention can also be achieved in the form of a computer data signal in which the program is embodied via electronic transmission and which is embedded in a carrier wave.

The present invention is not limited to the embodiments, but can be altered by a skilled person in the art within the scope of the claims. The present invention also encompasses, in its technical scope, any embodiment derived by combining technical means disclosed in differing embodiments.

### REFERENCE SIGNS LIST

1: safety checking system
10: living room
25: acceptance management section
30: control section (control device)
31: determining section
32: alerting section
33: communication control section
41: first sensor
42: second sensor
43: communication section
50: storage section
51: threshold

1. A safety checking system comprising:
a first sensor which senses, in a non-contact manner, biological information of a subject within a given sensing range;
a second sensor which detects, in a non-contact manner, the number of subjects within the sensing range of the first sensor; and
a control section which controls the first sensor and the second sensor, the control section being configured to cause, in a case where the second sensor detects that only one subject is present within the sensing range, the first sensor to measure biological information of the one subject.

2. The safety checking system according to claim 1, wherein:
in a case where the second sensor detects that only the one subject having been present within the sensing range has exited from the sensing range, the first sensor is caused to end the measurement of the biological information of the one subject.

3. The safety checking system according to claim 1 or 2, wherein:
in a case where a second sensor corresponding to a first sensing range and a second sensor corresponding to a second sensing range which is adjacent to the first sensing range detect that only the one subject having been present in the first sensing range has moved from the first sensing range to the second sensing range, (i) data of biological information of the one subject measured by a first sensor corresponding to the first sensing range and (ii) data of biological information of the one subject measured by a first sensor corresponding to the second sensing range are dealt with as a series of continuous pieces of data.

4. The safety checking system according to claim 3, wherein:
the second sensor has a detection range larger than the sensing range of the first sensor.

5. The safety checking system according to claim 4, wherein:
the first sensor measures at least either of a heart rate and a respiratory rate of the one subject.

6. A control device configured to control:
a first sensor which senses, in a non-contact manner, biological information of a subject within a given sensing range; and
a second sensor which detects, in a non-contact manner, the number of subjects within the sensing range of the first sensor, and
in a case where the second sensor detects that only one subject is present within the sensing range, the control device causing the first sensor to measure biological information of the one subject.

7. A living room comprising:
a first sensor which senses, in a non-contact manner, biological information of a subject within a given sensing range in the living room;
a second sensor which detects, in a non-contact manner, the number of subjects within the sensing range of the first sensor; and
a control device which controls the first sensor and the second sensor, the control device being configured to cause, in a case where the second sensor detects that only one subject is present within the sensing range, the first sensor to measure biological information of the one subject.

8. A safety checking method comprising:
a sensing step of causing a first sensor provided in a living room to sense, in a non-contact manner, biological information of a subject within a given sensing range in the living room;
a detection step of causing a second sensor provided in the living room to detect, in a non-contact manner, the number of subjects within the sensing range of the first sensor; and
a measurement step of causing, in a case where the second sensor detects that only one subject is present within the sensing range, the first sensor to measure biological information of the one subject.
